(19) United States
(12) Patent Application Publication
(10) Pub. No.: US 2002/0183762 A1
(43) Pub. Date: Dec. 5, 2002

(54) Bone anchor inserters and methods

(75) Inventors: Kimberly A. Anderson, Eagan, MN (US); Johann J. Neisz, Coon Rapids, MN (US); Gary A. Rocheleau, Maple Grove, MN (US); Robert E. Lund, St. Michael, MN (US)

Correspondence Address: AMS Research Corporation, Attention: Jeffrey J. Hohenshell, 10700 Bren Road West, Minnetonka, MN, 55343, US

(73) Assignee: AMS Research Corporation

(21) Appl. No.: 10/133,271

(22) Filed: Apr. 26, 2002

Related U.S. Application Data
(63) Continuation-in-part of application No. 29/151,465, filed on Nov. 9, 2001.
(60) Provisional application No. 60/295,328, filed on Jun. 1, 2001.
(60) Provisional application No. 60/295,330, filed on Jun. 1, 2001.
(60) Provisional application No. 60/311,776, filed on Aug. 10, 2001.

(51) Int. Cl. A61B 17/56

(52) U.S. Cl. 606/104

(57) ABSTRACT

Bone anchor inserters are described. The inserters may be used in novel methods. Kits that include the novel inserters are also described.

### CROSS REFERENCE TO RELATED APPLICATIONS

The present application is a continuation-in-part of U.S. Design patent application Ser. No. 29/151,465 filed Nov. 9, 2001 and claims priority to U.S. Provisional Application Serial No. 60/295,328 filed Jun. 1, 2001, and U.S. Provisional Application Serial No. 60/295,330 filed Jun. 1, 2001, and U.S. Provisional Application Serial No. 60/311,776 filed Aug. 10, 2001. The entire contents of the provisional patent applications and the design patent are herein incorporated by reference in their entirety.

### BACKGROUND

Bone anchors are used for a variety of surgical purposes. Orthopedic or soft tissue surgical procedures that utilize bone anchors are disclosed in U.S. Pat. Nos. 5,376,097, 5,572,342, 5,643,320, 5,741,282, and 5,980,558; percutaneous surgical procedures for treating incontinence are disclosed in U.S. Pat. Nos. 5,611,515, 5,836,314, 5,842,478 and 5,860,425; transvaginal surgical procedures for treating incontinence are disclosed in U.S. Pat. Nos. 5,972,000, 6,039,686, 6,053,935, 6,328,686, 6,322,492, 6,328,686 and 6,334,446; and surgical procedures for treating airway obstructions, sleep apnea and snoring are disclosed in U.S. Pat. No. 5,988,171. Other incontinence procedures and/or surgical instruments are described in U.S. Pat. Nos. 6,241,736 and 6,319,272.

Bone anchors include bone screws, staples and bone tacks. U.S. Pat. Nos. 5,520,700, 5,674,247, 5,807,403 and 5,873,891 describe various bone anchors. Some bone anchors include a hole or eyelet for threading a suture to associate the suture with the bone anchor.

Placing a bone anchor in a remote region of a patient presents a particular challenge for a surgeon. Often, the intended location of the screw is surrounded by sensitive, vulnerable tissue such as arteries, nerves and veins.

A variety of different surgical instruments have been developed to place a bone anchor. Some surgical instruments resemble a common screwdriver. These devices generally insert the bone anchor into the bone in a direction away from the surgeon. Other surgical instruments are capable of placing a bone anchor at a remote region of the body, such as on a posterior portion of a patient's pubic bone or the patient's mandible. These devices can insert the anchor into the bone in a direction toward the surgeon.

Bone anchors are particularly useful as a component of a suprapubic sling procedure for stress urinary incontinence. Some procedures use a transvaginal approach and place a bone screw or tack on a posterior surface of the pubic bone.

The Vesica™ Sling Kits, available from Microvasive (affiliated with Boston Scientific and Scimed), utilize a percutaneous suprapubic approach to place a bone anchor on the top of a patient's pubic bone. The percutaneous suprapubic approach involves a relatively large abdominal incision. Complications associated with this procedure include osteitis pubis and osteomyelitis. See Appell RA, The Use of Bone Anchoring in the Surgical Management of Female Stress Urinary Incontinence, World J. Urol. (1997); 15: 300-305; and Stram T R, Ablove R H, Lobel R W, Infectious Complications of Vaginal Wall Suburethral Slings with Suprapubic Bone Anchors, Intl. Urogynecology J. (2000); 11 (Suppl. 1): abstract 015.

Other surgical procedures utilize a power drill to drill a hole for subsequent implantation of a bone anchor on top of a patient's pubic bone. After an abdominal incision is made, the surgeon prebores a hole in the bone with a drill, removes the drill, introduces a seating device, seats the bone anchor screw and then threads a suture. This procedure is somewhat cumbersome and it is believed that surgeons can lose access to the bored hole or seated bone screw. As a result, some surgeons need to drill additional holes and/or seat additional bone screws.

The Precision Twist™ Transvaginal Anchor System (available from Microvasive of Boston Scientific, of Natick, Mass.) includes a manually powered device for placing a bone screw in the posterior portion of a patient's pubic bone. Squeezing a handle on the device causes the bone screw to rotate through the use of a resilient member and pawl mechanism. The device is inefficient in that it requires the user to squeeze the device multiple times in order to fully implant the screw. When the handle of the device is released after a squeeze, no rotation is imparted to the screw during the return stroke. Some users find the repeated squeezing awkward and difficult to use.

Control is believed to be another problem associated with the Precision Twist device. A user typically pulls the device against the pubic bone with one hand and squeezes the handle with the other. When both hands are used to deliver the screw, the surgeon cannot palpate structures to help ensure proper orientation of the screw relative to the bone. For example, some surgeons prefer to palpate the pubic bone to accurately place the screw relative to the periosteum, cortex and medulla of the pubic bone. It is believed that inaccurate placement of bone screws can lead to several complications, such as damage to the periosteum, increased infection rates (e.g. due to the damage), and lower suture pull out force for the sutures associated with the screw.

The Precision Tack™ Transvaginal Anchor System (available from Microvasive) is used to place a tack on the posterior portion of a patient's pubic bone. The tack includes a trocar tip, a floating crown and an eyelet for associating the tack with a suture. The tack is manually inserted into the bone by pulling on the handle of the insertion device. The handle is elongate along an axis that is substantially aligned with the direction of pull. Some users find it difficult to deliver the force required to seat the tack due to the shape and orientation of the handle.

It is believed that the tacks associated with the Precision Tack device afford a less secure anchor in the pubic bone than the anchor provided by a bone screw. It is also believed that the body tends to heal more efficaciously after bone screw placement than after the bone tack placement, and the nature of the force used to place a Precision tack is believed more damaging to surrounding tissues and structures, which can lead to complications. The tacks associated with the Precision Tack device are also believed to provide a less reliable suture anchor.

The In-Fast™ Sling System is available from American Medical Systems, Inc. of Minnetonka, Minn. The In-Fast System includes a single-use, battery-powered device that is capable of driving screws into bone. The inserter is shaped to place the screw on the posterior of the patient's pubic bone. The In-Fast Inserter utilizes a rigid shaft and gear system to accomplish a ninety-degree bend.

The In-Fast Inserter receives a screw and associated suture in a screw mount. The device inserts a screw along a substantially straight insertion axis. The distance along the insertion axis between the end of the screw mount and the distal end of the device is the Bone Clearance Length. The Bone Clearance Length is related to the amount of room needed for insertion of a screw into bone, and the amount of blunt tissue dissection endured by the patient. When the insertion device is used in a remote region of the body (e.g. on the posterior portion of the pubic bone), this translates into the amount of tissue disruption needed to insert the screw. The Bone Clearance Length of the In-Fast Inserter is about 0.87 inches.

The In-Fast Inserter includes a main body portion connected to the handle and a reduced neck portion. The shortest distance between the outer surface of the neck portion and the insertion axis is the Insertion Depth Length. The Insertion Depth Length is a constraint on the ability of the device to place a bone screw in remote regions the body. For example, during an incontinence procedure using a transvaginal approach, the Insertion Depth Length limits the placement of screws to locations (i.e. the height on the pubic bone) within the limit of the Insertion Depth Length. Any further insertion would be blocked by contact between the body (e.g. the bottom of the pubic bone) and the neck portion of the inserter device. The Insertion Depth Length of the In-Fast Inserter is about 1.34 inches. The length of the reduced neck portion is about 1 13/16 inches. The overall length of the In-Fast device is about eight inches.

While the In-Fast Inserter works well, it could be used more conveniently and efficiently in procedures involving obese or large patients, or patients with very small pelvic structures, or patients with scarring or other pelvic anomalies that can limit access.

### BRIEF SUMMARY

The present invention comprises novel surgical bone anchor inserter devices and novel methods that may involve such devices. In one aspect, the inserter includes a battery-powered device with a distal portion that is significantly reduced in size. For example, the Bone Clearance Length is reduced. The smaller size affords a smaller surgical incision, and less need to damage tissue to make room for the head of the inserter. This can translate into reduced surgical trauma, reduced infection rates, and faster patient recovery time.

The present invention also includes a motor powered device that includes an increased Insertion Depth Length. This affords access to even more remote regions and greater control over accurate placement of the bone anchor.

In another aspect, the present invention comprises a surgical device with a handle and operating elements that are sized, shaped and oriented to afford precise placement of a bone anchor at a predetermined position.

The present invention also includes efficient, manually powered bone anchor insertion devices. These devices include a handle that is movable in substantially opposite directions. The device is capable of imparting rotational force to a bone screw in both directions of movement of the handle.

In one aspect, the present invention comprises a surgical device for inserting a bone anchor. The device comprises a handle, a battery and motor assembly, a bone anchor deployment portion with a bone anchor mount that is operatively associated with the battery and motor assembly. The Bone Clearance Length of the device is less than about 0.8 inches, more preferably less than about 0.7 inches.

In another embodiment comprises a surgical device having a handle, a battery and motor assembly, and a bone anchor deployment portion. The Insertion Depth Length (IDL) in this embodiment is more than about 1.4 inches, preferably more than about 1.5 inches and even more preferably the IDL is greater than about 1.8 inches.

In another embodiment, the inserter comprises a drive including a flexible drive shaft, means for rotating the flexible drive shaft, and a bone anchor deployment portion with a bone anchor mount that is operatively associated with the flexible drive shaft. The inserter comprises a main body associated with the handle, and an extension portion projecting from the main body. The extension portion includes a body projection portion that projects from the main body, a bend and a portion that extends in a direction that is at an angle (e.g. preferably approximately ninety degrees) relative to the direction of projection of the body projection portion. Preferably, the bend comprises an arc having a radius of less than about 1.5 inches, more preferably the arc has a radius of less than about 1.2 inches. Preferably, the extension portion has a length that is more than about 1⅞ inches.

The drive preferably includes a drive bevel gear associated with the flexible drive shaft. The bone anchor mount preferably includes a driven bevel gear. When a flexible drive shaft is used, the drive bevel gear preferably includes compensation means-for compensating for a reduction in length of the flexible drive shaft during rotation of the flexible drive shaft under load conditions. The compensation means comprises a channel in the drive bevel gear that affords axial movement of the drive shaft relative to the drive bevel gear.

A variety of different drive mechanisms are contemplated herein including powered drives (e.g. a battery powered motor) and manually powered drives. The drives can include rigid shafts, flexible shafts, stepped gears, belts and the like.

In one embodiment, the present invention comprises a manually powered surgical instrument for placing a self-tapping bone screw and an associated suture on the posterior portion of a pubic bone during a surgical procedure for treating incontinence. The instrument comprises a handle, and a manually movable lever having an end. The end of the lever is movable relative to the handle between a release position with the end of the lever spaced from the handle, and a squeezed position with the end of the lever spaced closer to the handle than in the release position. The surgical instrument includes a body portion, and a screw mount for receiving the self-tapping screw. The screw mount is rotatable to rotate the self-tapping screw into pubic bone in a delivery rotation direction.

The components of the manually powered surgical instrument are sized, shaped and arranged to be placed through a vaginal incision. The manually powered instrument includes a drive operatively associated with the lever to rotate the screw mount in the delivery rotation direction both when the lever moves from the release position toward the squeezed position and when the lever moves from the squeezed position toward the release position. This increases the convenience and efficiency of the device and affords greater ease of use.

Preferably, the manually powered device includes a pair of finger flanges for the surgeon to grasp to orient the device relative to the pubic bone. In one species of surgical device, the drive includes a double ratchet mechanism.

In another manually powered embodiment, the instrument comprises a handle, and a manually movable lever, a body portion with a pair of finger flanges, and a bone screw deployment portion that includes a screw mount for receiving the self-tapping screw. The screw mount is rotatable to rotate the self-tapping screw into a pubic bone in a delivery rotation direction. In this embodiment, the handle and the bone screw deployment portion project from a same side of the body portion. This embodiment includes a drive operatively associated with the lever to rotate the screw mount in the delivery rotation direction.

In another aspect, the present invention comprises novel drive assemblies for bone anchor insertion devices. The novel drives may be utilized in powered and manual versions of the device. In one embodiment, the present invention comprises a drive including a flexible drive shaft that is bent about its axis about a radius, means for rotating the flexible drive shaft, a drive bevel gear capable of being rotated by the flexible drive shaft, and a bone anchor deployment portion with a bone anchor mount that includes a driven bevel gear capable of engaging the drive bevel gear to rotate the bone anchor mount in a screw delivery direction. The drive bevel gear preferably includes compensation means for compensating for a reduction in length of the flexible drive shaft during rotation of the flexible drive shaft under load conditions.

In another aspect, the present invention comprises a preferred bone anchor deployment portion having a distal side and a proximal side opposite the distal side. The proximal side has a bone anchor mount and optional bone anchor shield. The distal side has a palpable, irregular surface in substantially the region where a bone anchor delivery axis intersects the distal side of the anchor deployment portion. The irregular surface provides tactile information to the surgeon concerning the precise location of the bone anchor delivery axis. This feature contributes to accurate bone anchor placement.

In another aspect, the present invention includes novel surgical methods. The above-identified novel surgical instruments and components may be utilized in the novel methods according to the present invention.

In another aspect, the present invention comprises novel surgical kits. The kits may include an novel surgical inserter as described above. The kits may also be used in the surgical methods of the present invention.

These and other advantages of the invention are more fully shown and described in the drawings and detailed description of this invention, where like reference numerals are used to represent similar parts. It is to be understood, however, that the drawings and description are for the purposes of illustration only and should not be read in a manner that would unduly limit the scope of this invention.

### BRIEF DESCRIPTION OF THE DRAWINGS

Other features and advantages of the present invention will be seen as the following description of particular embodiments progresses in conjunction with the drawings, in which:

FIG. 1 is a perspective view of a surgical instrument according to one aspect of the present invention;

FIG. 2 is a right side view of the instrument of FIG. 1;

FIG. 3 is a top view of the instrument of FIG. 1;

FIG. 4 is a bottom view of the instrument of FIG. 1;

FIG. 5 is an end view of a proximal portion of the instrument of FIG. 1;

FIG. 6 is an end view of a distal portion of the instrument of FIG. 1 with portions broken away to emphasize details;

FIG. 7 is a schematic view of a distal portion of the surgical instrument of FIG. 1 as it inserts a bone screw in a pubic bone of a patient;

FIG. 8 is a perspective view of a distal portion of the surgical instrument of FIG. 1 as it inserts a bone screw, with portions broken away to illustrate a posterior portion of the pubic bone;

FIG. 9 is a perspective view showing a sling held in place by sutures associated with bone screws;

FIG. 10 is a perspective view of the surgical instrument of FIG. 1 showing the instrument partially disassembled;

FIG. 11 is a side view of the surgical instrument of FIG. 1 with a cover of the body removed to illustrate internal elements;

FIG. 12 is a side view of the handle of the instrument of FIG. 1 with the cover removed to illustrate details of components within the handle;

FIG. 13 is a sectional view of a distal portion of the instrument of FIG. 1;

FIG. 14 is an enlarged sectional view of the portion of FIG. 13 identified approximately by the broken line circle;

FIG. 15 is a sectional view of a shield for optional use with the present invention;

FIG. 16 is an enlarged side view of a drive bevel gear shown in FIG. 13;

FIG. 17 is an enlarged sectional view of a component that comprises a driven bevel gear and an anchor mount, shown in a different section in FIG. 13;

FIG. 18 is an enlarged perspective view of a control lever element of the device of FIG. 1;

FIG. 19 is a top view of a kit according to one aspect of the present invention;

FIG. 20 is a side view of an alternative embodiment of surgical instrument according to the present invention, with portions broken away to illustrate details;

FIG. 21 is a side view of an alternative embodiment of surgical instrument according to the present invention, with portions broken away to illustrate details;

FIG. 22 is a side view of an alternative embodiment of surgical instrument according to the present invention, with a side removed to illustrate internal details;

FIG. 23 is a side view of an alternative embodiment of surgical instrument according to the present invention, showing a direction of adjustment with an arrow;

FIG. 24 is a side view of an alternative embodiment of surgical instrument according to the present invention, with portions broken away to schematically illustrate details of a manually powered drive assembly;

FIG. 25 is a schematic perspective view of one embodiment of a linear to rotational force converter according to an aspect of the present invention;

FIG. 26 is a schematic perspective view of another embodiment of linear to rotational force converter according to an aspect of the present invention;

FIG. 27 is a schematic view of a micro switch circuit for use with an embodiment of the present invention;

FIG. 28 is a schematic view of another element of a microswitch according to the present invention;

FIG. 29 is a schematic view of a gearbox for use in an embodiment of the present invention;

FIG. 30 is a schematic perspective view of another embodiment of drive for optional use in an instrument according to the present invention;

FIG. 31 is a side sectional view of another embodiment of surgical instrument according to the present invention;

FIG. 32 is a bottom sectional view of the surgical instrument of FIG. 31;

FIG. 33 is a perspective view of the surgical instrument of FIG. 31, with portions broken away to illustrate details; and

FIG. 34 is a side view of a component used in the embodiment of FIGS. 31-33.

### DETAILED DESCRIPTION

The following description is meant to be illustrative only and not limiting. Other embodiments of this invention will be apparent to those of ordinary skill in the art in view of this description.

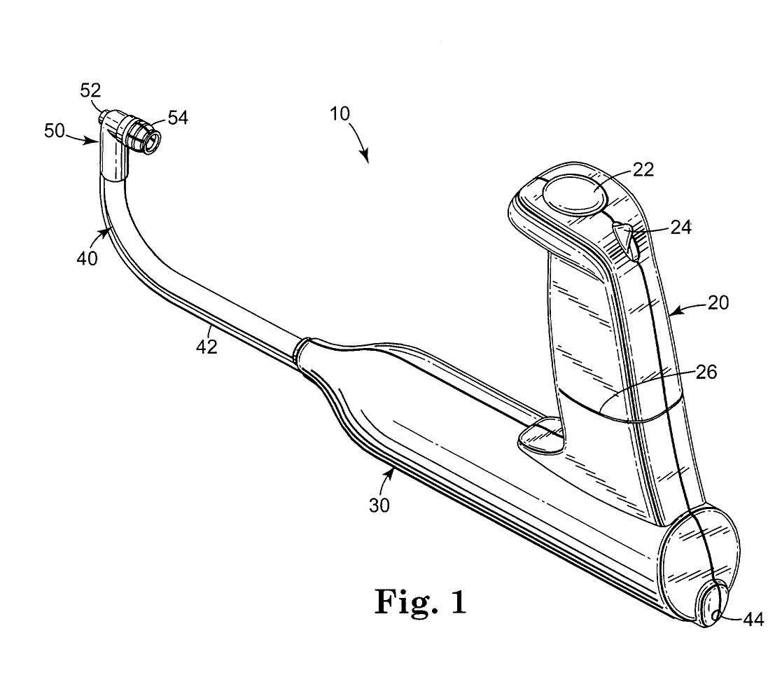

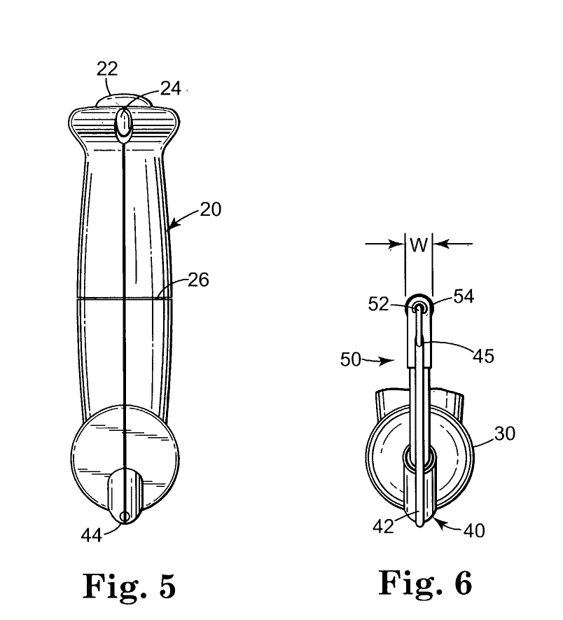

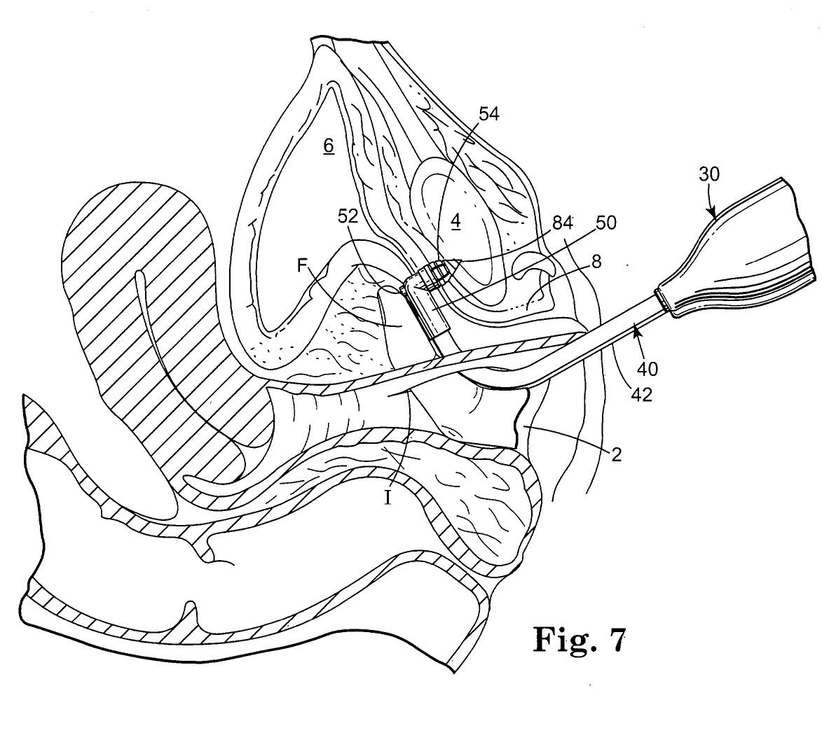

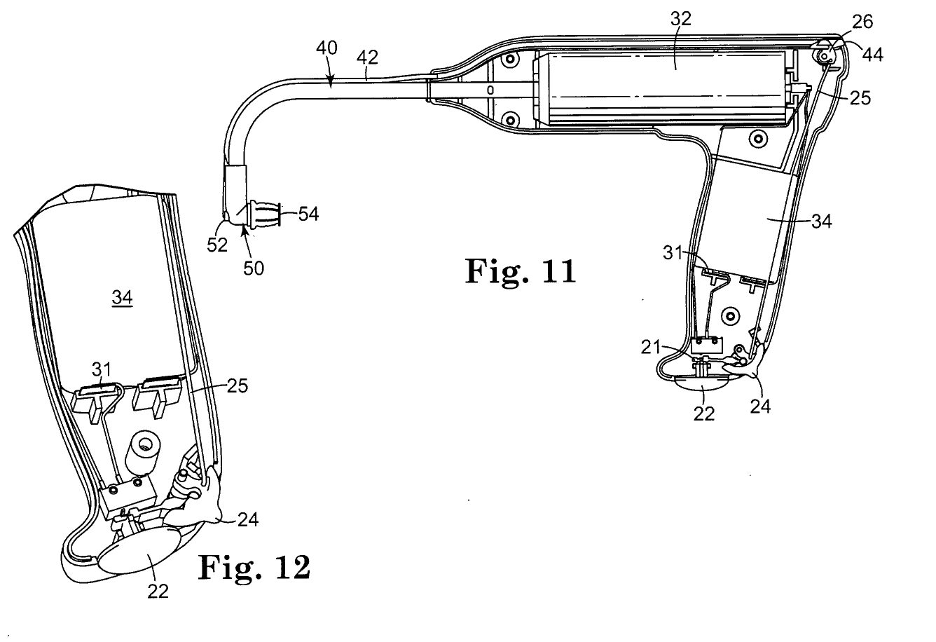

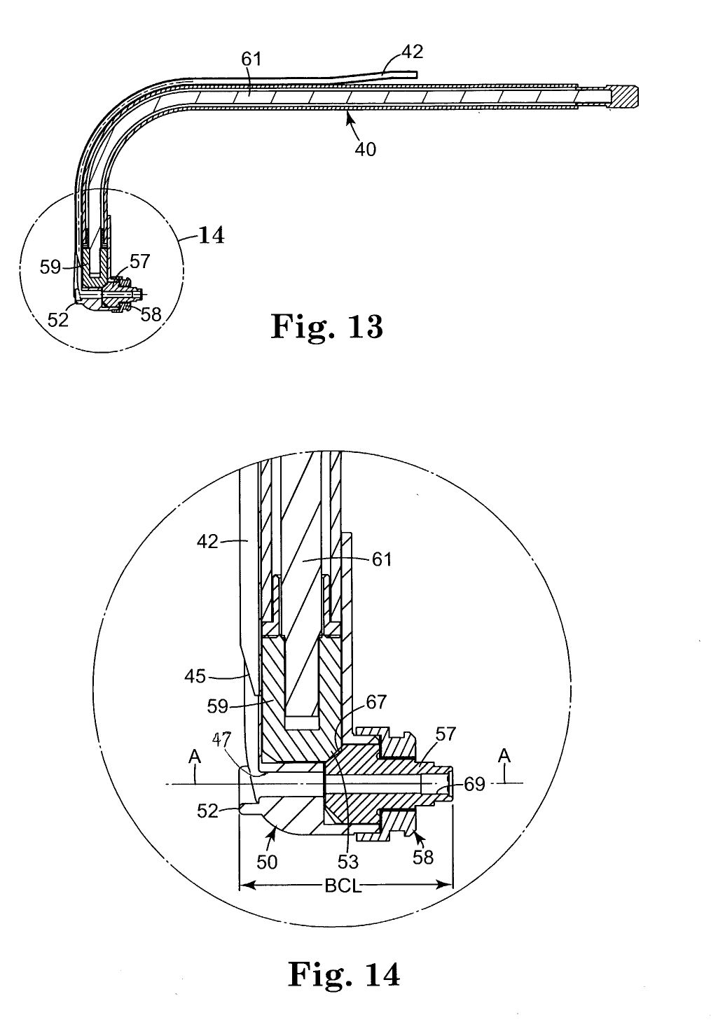

FIGS. 1 through 6 and 10 through 18 illustrate components of an embodiment of surgical instrument 10 for delivering a bone anchor according to the present invention. The instrument 10 is particularly suitable for being inserted through a vaginal incision and driving a bone anchor (see FIG. 7) into the posterior portion of a pubic bone 4 of a patient to treat stress urinary incontinence (SUI) diagnosed with urethral hypermobility or intrinsic sphincter deficiency

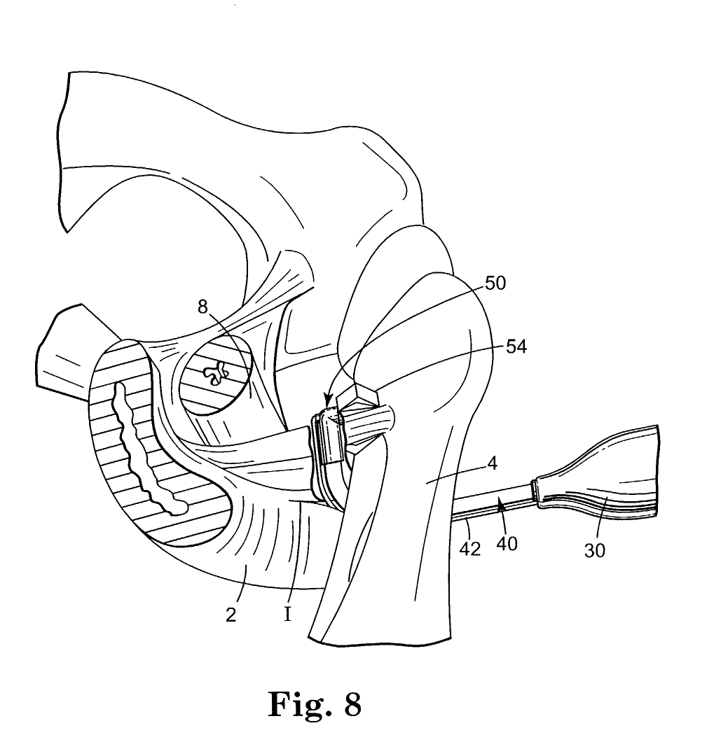

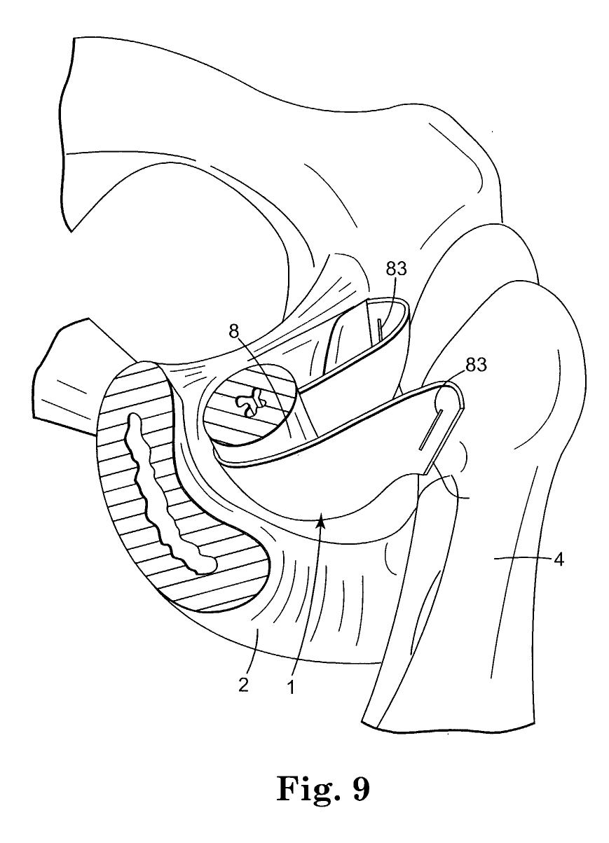

FIGS. 7 and 8 schematically illustrate the use of a surgical instrument 10 during a surgical procedure for treating female incontinence. The figures generally show the location of elements of the device 10 relative to anatomical locations such as the bladder 6, urethra 8, vaginal surgical incision I, and vagina 2. FIG. 7 illustrates a bone anchor 84 after it is placed in the pubic bone. It also illustrates a portion of the inserter 10 situated just before it is used to insert the bone anchor 84. The bone anchor 84 typically includes a suture associated therewith. FIG. 9 illustrates a sling 1 associated with bone screws via sutures 83.

The instrument 10 can be used to treat both men and women. Although the invention as disclosed herein generally refers to SUI, treatment of other urological disorders, such as mixed incontinence, urge incontinence (e.g. by suture anchoring an electronic implant device), and concomitant procedures such as prolapse (e.g. vaginal), enteroceles (e.g. of the uterus), and rectoceles are also within the scope of the present invention. Other non-urological disorders such as orthopedic procedures, soft tissue anchoring procedures (particularly remote applications), spinal procedures, and surgical procedures for treating airway obstructions, sleep apnea and snoring are also included within the scope of the present invention. It is contemplated that the present invention may also be utilized in conjunction with other pelvic procedures, such as, but not limited to, procedures for addressing cystocele prolapse, pelvic floor defect repair, and anatomic hypermobility.

The instrument 10 is particularly suitable for use during a sling procedure for treating incontinence. Any suitable sling material may be used according to surgeon preference. The sling 1 (FIG. 9) may comprise a synthetic material or a non-synthetic material or a hybrid combination of such materials. As used herein, the term “sling” is used generally to include a wide variety of shapes and sizes, materials and treatments. While the sling 1 is preferably rectangular for treating SUI in females, other shapes are also contemplated. Depending on the treatment addressed (e.g. to provide hammock support for the bladder or bladder neck, or to address a rectocele, enterocele or prolapse) the sling may be any of a wide variety of shapes. For example, the sling 1 may comprise any of the slings described in U.S. patent application Ser. Nos. 09/917,443, filed Jul. 27, 2001; and 10/005,837, filed Nov. 9, 2001 and PCT International Publication No. WO02/19945, the entire contents of each of which are herein incorporated by reference.

The precise, final location of the sling 1 will depend on a variety of factors including the particular surgical procedure(s) performed, and any preconditions of the patient such as scar tissue or previous surgeries. For example, it may be preferred to place the sling 1 in close proximity to, but not in contact with, a mid portion of the urethra 8 to treat incontinence. Alternatively, the sling 1 may be placed closer to the bladder neck for a bladder neck suspension procedure. In a male patient, the sling 1 may be placed proximate, but not in contact with the bulbar urethra. In another procedure, the sling 1 is not used. Instead, the sutures associated with the bone screws are used to suspend pelvic tissue (e.g. the upper portions of the vagina, lateral to the urethra, or endopelvic fascia) to address the urological disorder.

The device 10 comprises a handle 20, a battery 34 and motor assembly 32, and a bone anchor deployment portion 50. Referring to FIG. 14, the bone anchor deployment portion 50 has a first end portion with a raised, palpable surface 52 with a suture management opening, and a second end portion with a bone anchor mount 69 that is operatively associated with the battery 34 and motor assembly 32.

The battery 34 may be rechargeable or disposable. For example, the battery 34 may comprise a lithium battery such a commercially available six-volt lithium battery. Suppliers include Polaroid, GP (iec size 2CR5) or any other supplier of commercial batteries.

The motor 32 may comprise any suitable drive motor. Preferably, the motor comprises a low speed, rotational motor. More preferably, the motor provides a speed less than about 100 rpm, more preferably, less than 60 rpm, even more preferably, less than about 30 rpm. The torque provided by the instrument 10 is preferably less than about 10 kg-cm, more preferably less than about 5 kg-cm, more preferably about 4.2 kg-cm. For example, the motor 32 may comprise a type Alcatel-G30.1, 4V/PLG30, I=91.12:I, SW5 Bore motor, generally available from Dunkermotoren of Germany (spec. no.8885001490).

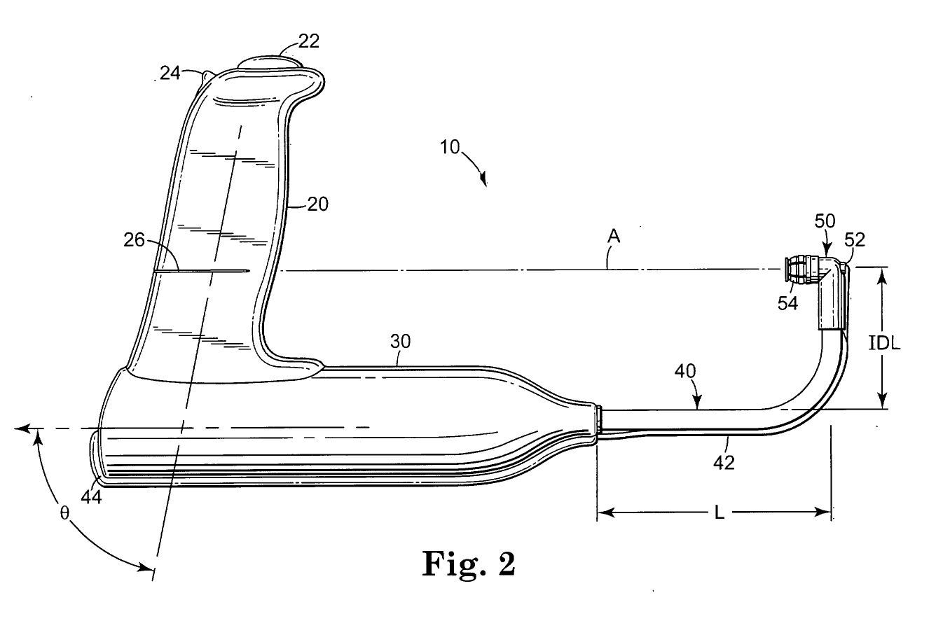

The bone anchor mount 69 is sized and shaped to receive a bone anchor. Preferably, the bone anchor is a self-tapping screw 84 (see FIG. 7), but it is contemplated that the present invention may be used in conjunction with bone tacks or bone staples and the like. The bone anchor deployment portion 50 is capable of driving the bone anchor 84 along a bone anchor delivery axis A (see FIG. 2). Referring again to FIG. 14, the distance between an end of the bone anchor mount 69 and the first end portion of the bone anchor deployment portion comprises the Bone Clearance Length BCL. This length is preferably as small as possible to avoid tissue disruption or trauma, preferably the BCL is less than 0.8 inches, more preferably, the BCL is less than about 0.75 inches and even more preferably the BCL is less than about 0.7 inches. In a preferred embodiment, the BCL is about 0.675 inches.

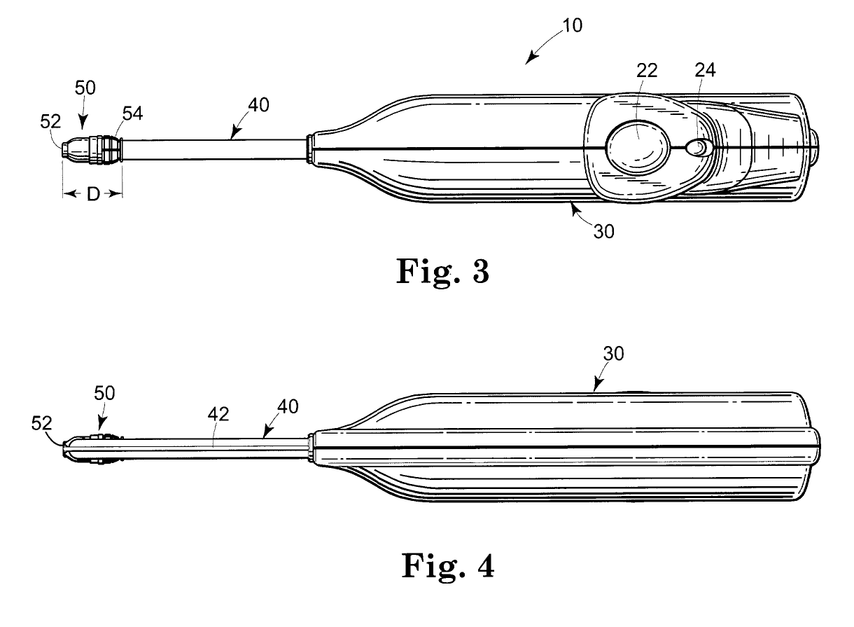

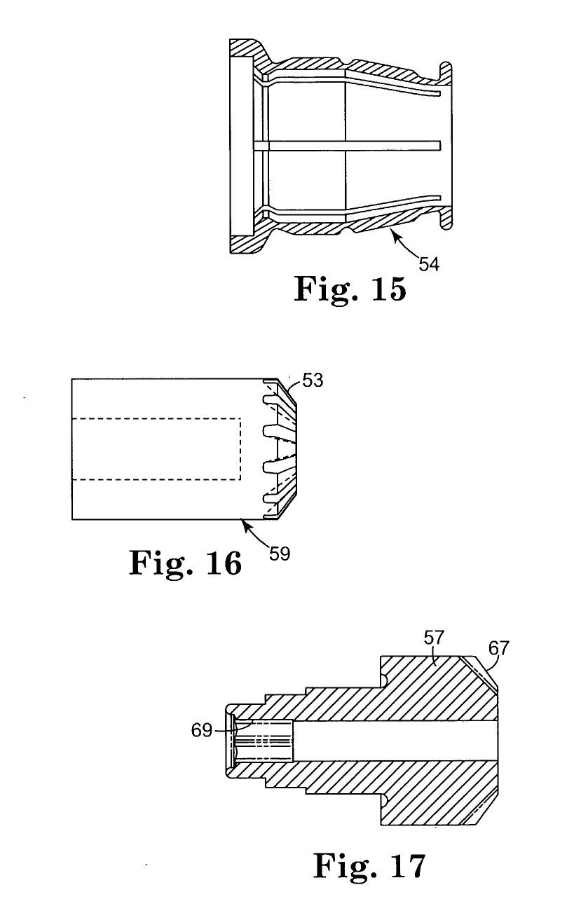

Optionally, a deflectable shield 54 (FIG. 15) may be placed about the bone anchor mount 69 to protect the bone anchor 84. Some surgeons may prefer to remove the shield 54 and use the instrument 10 without the shield 54. The effective Bone Clearance Length BCL may be slightly longer when the shield 54 is attached. For example, the length D shown in FIG. 3 may be slightly longer than the BCL. Preferably the length D is less than about 1 inch, more preferably, it is less than about 0.84 inches.

The collapsible shield 54 may be constructed from any suitable material. Suitable materials include polymerics, plastics, thermoplastic elastomers, silicone, PET, PETG, rubbers, vinyl, acrylic, latex, nylon and thermoset materials. For example, the shield 54 may be constructed from polypropylene, available from Exxon as PP 9074 (MED). Alternatively, the instrument may include a protective cap such as that shown and described in U.S. Pat. No. 6,053,935.

The width W (FIG. 6) of the bone anchor deployment portion 50 is preferably as small as possible. Preferably, the width W is less than 0.8 inches, more preferably, it is less than 0.7 inches, more preferably, it is less than 0.4 inches. In one embodiment, the width W is about 0.36 inches.

The instrument 10 includes a main body 30 associated with the handle 20. An extension portion 40 projects from the main body 30 in a first direction (e.g. along the axis of the main body 30). Referring to FIG. 2, the extension portion 40 includes a body projection portion that projects from the main body 50, and a bend. Preferably, the extension portion 40 is a substantially tubular shape with a substantially circular cross section with a diameter less than 0.69 inches, more preferably, less than about 0.29 inches. The body projection portion has a length L along the axis of the main body 50 of more than about 1⅞ inches, more preferably, the length L is more than about 2 inches and even more preferably, the length is more than about 3 inches. In a preferred embodiment, the length L is about 3.25 inches to the back of the anchor delivery portion of the device. The overall length of the surgical instrument 10 is preferably greater than 8 inches and more preferably greater than about 10 inches. In a preferred embodiment, the overall length of the device is about 10.4 inches.

The main body 30 may have a cover constructed from a medical grade or biocompatible material. Suitable materials include steels and polymers such as polycarbonate and nylon (e.g. glass filled). For example, the cover of the main body may be constructed from Makrolon polycarbonate, #2485.

Preferably, a portion is included in the extension portion 40 that ultimately extends at an angle (preferably approximately ninety degrees) relative to the direction of projection of the body projection portion. As can be seen in FIG. 2, the device includes a substantially 90-degree angle. Alternatively, the bend need not be so severe as to result in a 90-degree angle. Any angle between 0 and 90 degrees is within an aspect of the present invention. Preferably, the bone anchor deployment portion 50 is oriented to insert a bone anchor 84 in a direction that is substantially toward the user of the device 10. Alternatively, other directions of insertion are contemplated. For example, in some male sling procedures, screws are placed on the descending pubic ramus, just below the pubic symphysis (e.g. the InVance Male Sling Procedure, available from American Medical Systems, of Minnetonka, Minn.). In such an application, the inserter of the present invention can have a bend of much less than ninety degrees (and preferably in the direction opposite to the direction of the bend of FIG. 2). In this embodiment, the inserter may insert the screw in a direction substantially away from the surgeon.

Referring again to FIG. 2, the extension portion 40 has an outer surface. The distance between the outer surface of the extension portion 40 and the bone anchor delivery axis A is the Insertion Depth Length IDL. The IDL length is preferably greater than about 1.4 inches, more preferably, the IDL is greater than about 1.5 inches and even more preferably the IDL is greater than about 1.8 inches. In a preferred embodiment, the Insertion Depth Length is about 1.875 inches.

The handle 20 includes indicia means 26. Indicia 26 are substantially in the region where a straight line drawn substantially along the bone anchor delivery axis A would intersect the handle 20. The indicia help orient the user and assist the user in placing the bone screw in a direction that is substantially perpendicular to the bone surface. When the instrument 10 is used during a transvaginal procedure (FIG. 7), the distalmost parts of the device may not be visible. The indicia 26 can help the surgeon properly orient the device relative to the pubic bone 4 (e.g. identified by palpation of the pubic bone through the abdomen or through the vaginal incision) and help ensure proper placement of the screw 84.

Several different indicia means are contemplated herein. As depicted, the indicia means 26 comprise a line on the handle. The line can be painted, printed or the like. The indicia can comprise a channel or groove in the handle, or a raised or embossed portion. Alternatively, the indicia can comprise a pin or rod projecting from the handle in the direction of the line 26 in the figures (e.g. at the level of the axis A). Optical embodiments of the indicia may also be utilized. For example, the optical embodiment may include an LED or similar light source that can project a spot on the patient's abdomen at substantially the location of the axis A.

The second end portion of the deployment portion 50 preferably faces the handle 20 and the first end portion is opposite the second end portion. The first end portion has an irregular surface 52 (FIG. 14) in substantially the region where the bone anchor delivery axis A would intersect the first end portion of the anchor deployment portion 50. The raised portion 52 is palpable so that a surgeon may place a finger F (FIG. 7) against this surface to afford tactile feedback concerning the location of the axis A. This information can assist some surgeons in precise placement of the screw 84 in the pubic bone 4.

The handle 20 is preferably canted relative to the main body 30. Referring again to FIG. 2, the handle 20 has a longitudinal axis, and the main body 30 has longitudinal axis. The included angle theta (Θ) between the two axes is preferably between about seventy and about eighty nine degrees. More preferably, the angle is approximately eighty degrees.

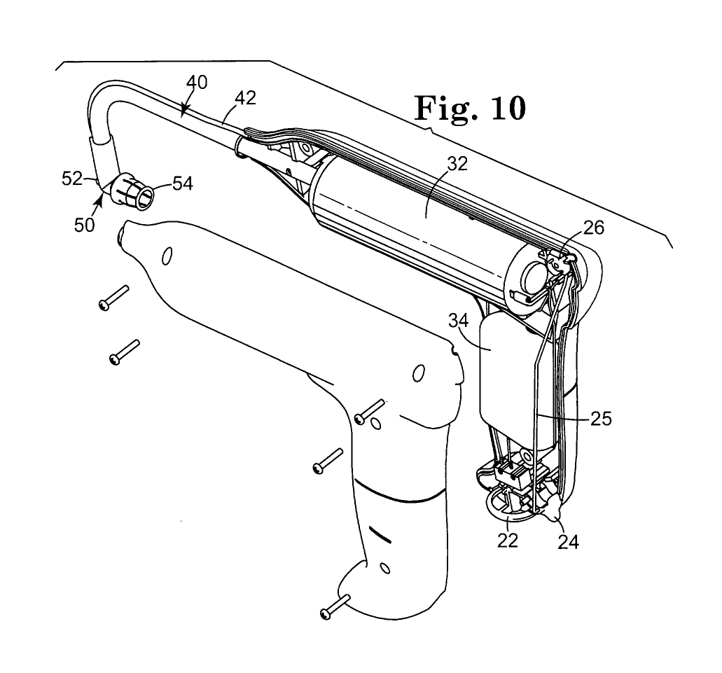

The surgical instrument 10 preferably comprises a battery powered drive, but manual, plug-in, ultrasonic motor, hydraulic, gas, and pneumatic embodiments are also contemplated herein. One example of a powered drive is shown in FIGS. 10-14. The instrument includes an activation button 22 (e.g. of a polycarbonate/ABS blend material). The activation button 22 can be depressed to activate switch 21. When the user depresses the activation button 22, current can flow through contact 31 and energize the motor 32. When the motor 32 is energized, it rotates flexible drive shaft 61.

In one embodiment, the present invention includes a novel flexible drive shaft 61 for transferring energy through a substantially 90-degree bend. The flexible drive shaft 61 should be sufficiently flexible to bend to the shape shown in the FIGS. 1-14, yet should have sufficient strength to transmit torque and rpm's sufficient for screwing a bone screw into bone. For a motorized version of the instrument 10, the flexible drive shaft 61 can be constructed from carbon steel, stainless steel, multilayered wound carbon steel, NiTi alloys, or the like. For example, the drive shaft 61 can be constructed from 300 series stainless steel with an ultimate torsional stiffness of about 25 lb-in (wind direction) and 17 lb-in (unwind direction) and a torsional deflection of about 25.5 deg/ft/lb-in.

The drive shaft 61 is associated with a drive bevel gear 59 (FIG. 16) with drive teeth 53. The drive shaft 61 rotates (e.g. clockwise) the bevel gear 59 about the axis of the bevel gear 59. The flexible shaft 61 has a tendency to shorten in length during rotation under a load. Referring to FIG. 14, the drive shaft is placed in a channel in the bevel gear 59. The cross sectional shape of the channel is complementary with the cross sectional shape of the shaft 61 in this region. For example, the cross sectional shapes of each may be polygonal or hexagonal (e.g. a regular hexagonal shape). Preferably, the end of the drive shaft 61 is not firmly affixed to the channel. Instead, the channel allows the drive shaft 61 to move axially within the channel during application of the load to the drive shaft 61.

The drive teeth 53 of the bevel gear 59 are adapted to engage complementary shaped driven teeth 67 on driven bevel gear 57. In one aspect of the present invention (e.g. the embodiment shown in FIG. 14), the driven bevel gear 57 and the anchor mount 69 comprise a unitary component. For example, for a self-tapping bone screw 84, the anchor mount 69 may comprise hexagonal shaped surfaces for receiving complementary surfaces on the base of the screw 84. The gears 59 and 57 may be constructed from any suitable material, such as, for example, stainless steel (e.g. 17-4 PH SST hardened to H900).

Preferably, the driven bevel gear 57 also includes a passageway 47 therethrough (FIG. 14) so that a suture associated with the bone anchor (e.g. screw 84) may be threaded from one side of the bone anchor deployment section 50 to the other side.

The instrument 10 also preferably includes a suture management feature. The instrument 10 includes a suture management tube 42 along a major portion of the length of the extension portion 40. The tube 42 preferably has an opening 45 proximate the deployment portion 50 and extends into the housing of the body 30. As seen in FIG. 6, there is preferably a trough or channel proximate tactile surface 52. The channel is preferably wide enough to receive a suture, but small enough so that a surgeon's finger is unlikely to pinch the suture. An internal suture passageway also extends along the length of the main body 30 terminating in opening 44 (FIG. 1). The passageway can optionally be integral with the cover material. The suture management feature helps prevent the suture 83 (FIG. 9) from becoming tangled or caught on body structures. Other suture management embodiments are described in U.S. Provisional Application Serial No. 60/311,776 filed Aug. 10, 2001.

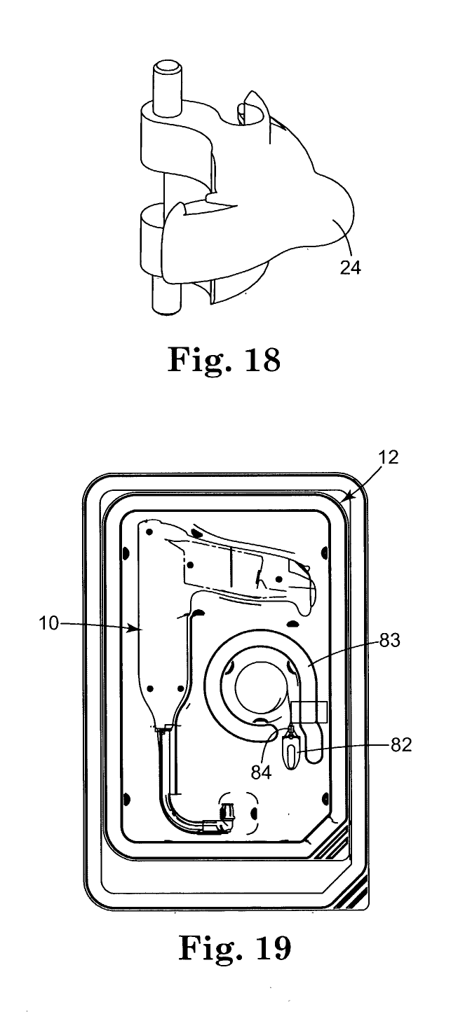

The instrument 10 also preferably includes a lockout lever 24 (FIG. 18). The lever 24 is movable between a locked position and a released position. In the locked position, the lever preferably blocks depression of the activation button 22. When activation button 22 is blocked, the battery 34 cannot energize the motor 32 and rotate the drive shaft 61.

The lockout lever 24 is preferably operatively associated with extension 25 and suture engagement surface 26. When the lockout lever 24 is in the lockout position, the extension 25 places the suture engagement surface 26 in a position to engage a suture within the internal passageway (e.g. exiting the body 30 at opening 44) to prevent movement of the suture.

When the lockout lever 24 is moved to the release position, the extension 25 spaces the suture engagement surface 26 from the suture within the internal passageway. In this position, the suture is free to move relative to the body 30. The activation button 22 is also free to be depressed when the lockout lever 24 is in the release position. Preferably, the handle 20 includes indicia for informing a user of the position of the lockout lever 24.

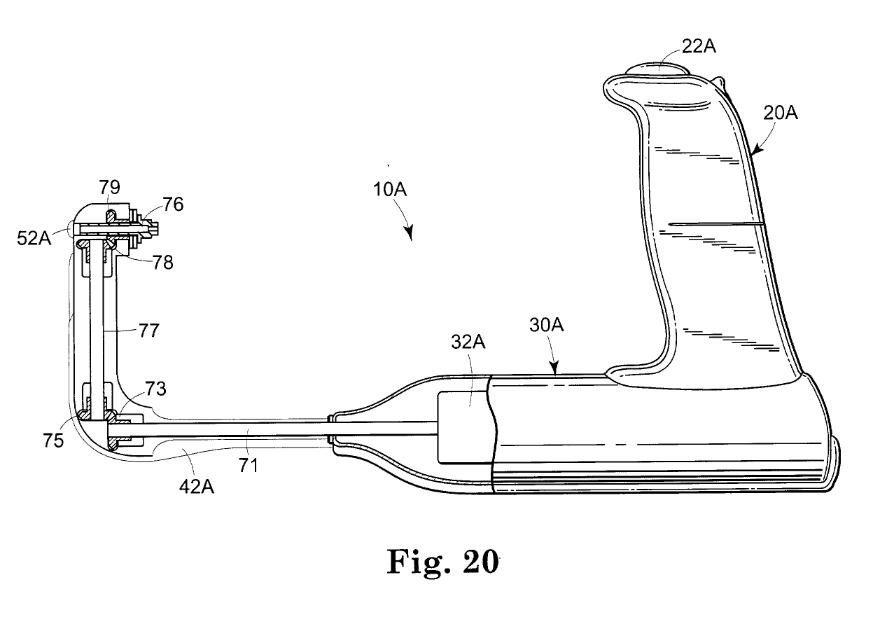

A plurality of different types of drives are suitable for use in the present invention. Any suitable combination of gearing and drive mechanisms may be utilized including spur gears, helical gears, double helical gears, worm gears and crossed axis helical gears. FIG. 20 illustrates a surgical instrument 10A having a handle 20A, suture management tube 42A, activation button 22A, main body 30A and motor 32A. This device includes a plurality of substantially straight shafts 71 and 77 (e.g. constructed from a rigid or substantially non-flexible material), and a plurality of bevel gears 73, 75, 78, 79 and an anchor mount 76. The instrument 10A also includes a palpable, irregular surface 52A that is raised or recessed (not shown), and indicia on the handle 20A.

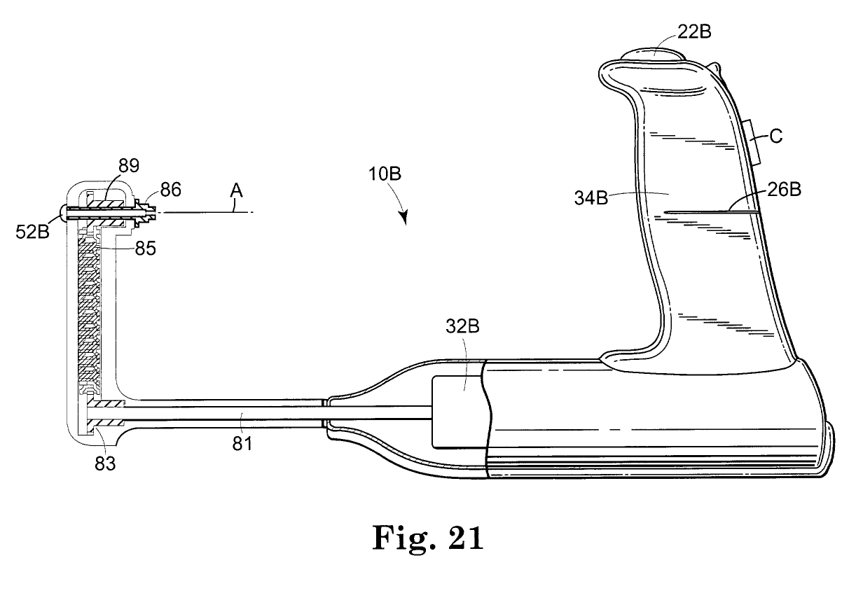

FIG. 21 illustrates another embodiment of surgical instrument 10B. The instrument 10B comprises a handle 34B with indicia 26B, activation button 22B, irregular surface 52B, and motor 32B. The handle 34B also includes a port C where the device may be plugged in to recharge a rechargeable battery or to power the motor 32B. The drive of this device includes a rotatable shaft 81 connected to a drive gear 83, a drive belt 85 (e.g. a chain transmission, timing belt, bead belt, metallic ribbon or toothed belt) and a driven gear 89 with anchor mount 86.

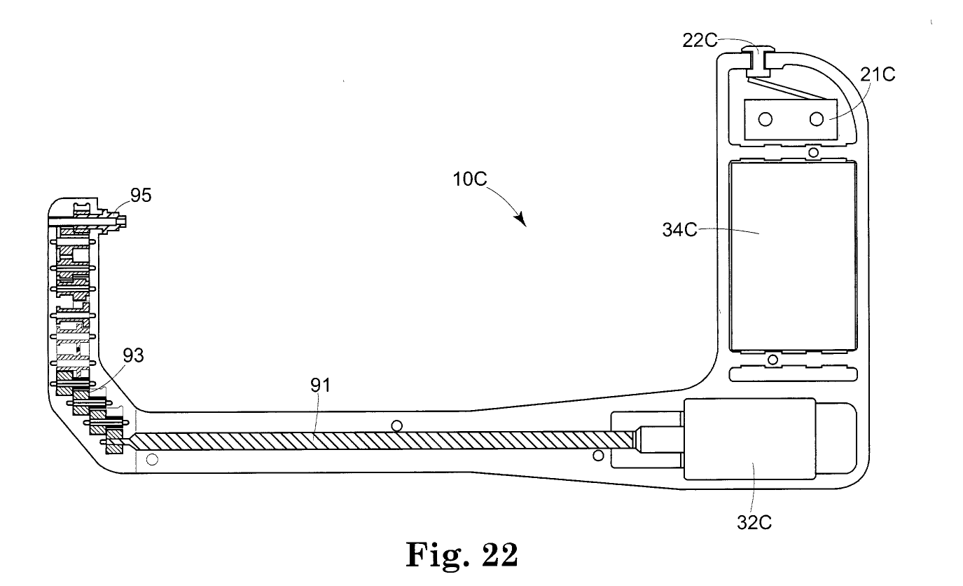

FIG. 22 illustrates another embodiment of surgical driver 10C according to the present invention. The driver 10C includes an activation button 22C, switch 21C, battery 34C and motor 32C. The drive of the instrument includes a substantially rigid drive shaft 91 and a plurality of stepped gears 93. The gears 93 can be sized and shaped to control the rpms and torque of the anchor mount 95. This embodiment provides a smaller head profile for the device 10C without using a flexible drive shaft. Suitable materials for the drive shafts include, but are not limited to tungsten and steel. Suitable materials for the gears include steel, tungsten or ceramic materials.

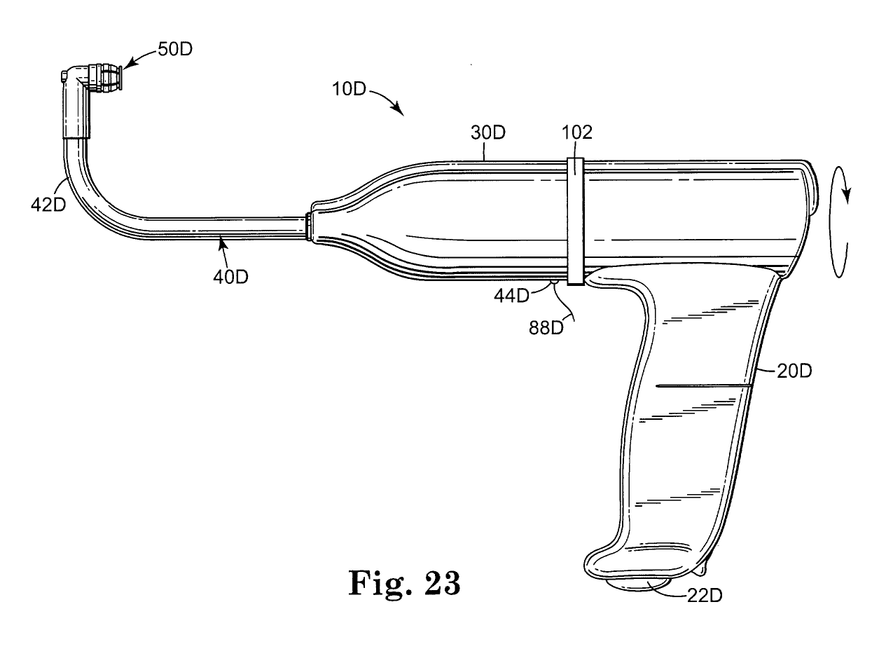

The surgical instruments according to the present invention are preferably C-shaped, but other configurations are within the scope of the present invention. FIG. 23 illustrates an embodiment of surgical driver 10D that is not C-shaped (as depicted). The driver 10D preferably includes a repositionable handle. The surgical driver 10D includes a projecting portion 40D, suture management tube 42D, suture opening 44D, suture 88D, anchor deployment portion 50D and handle with activation button 22D. The instrument 10D includes an adjustment means 102 that may be utilized to reposition the handle relative to the deployment portion 50D (e.g. in the direction of the arrow in FIG. 23). The adjustable handle instrument 10D may be especially useful for procedures with obese patients, or patients with anatomical structures that would make a C-shaped drive device difficult to use.

Optionally, the means 102 can be used to separate a disposable portion 30D of the instrument from a reusable portion 20D of the instrument 10D. For example, components of the device 10D that can withstand sterilization (e.g. the heat of a steam sterilization cycle, or the corrosive effect of ethylene oxide gas) may be placed in the reusable portion 20D, while elements of the instrument 10D that are adversely affected by sterilization may be positioned in the disposable portion of the device.

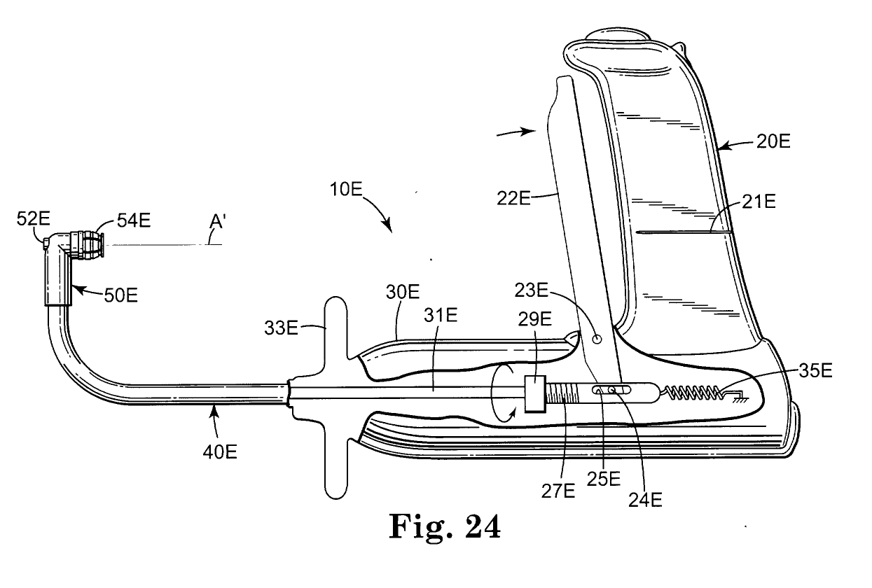

FIG. 24 illustrates a manually powered surgical instrument 10E for placing a self-tapping screw and an associated suture on the posterior portion of a pubic bone 4 during a surgical procedure for treating incontinence. The instrument 10E is sized, shaped and arranged to be placed through a vaginal incision.

The instrument 10E comprises a handle 20E, and a manually movable lever 22E having an end. The end of the lever 22E is movable (e.g. see the arrow in FIG. 24) relative to the handle 20E between a release position with the end of the lever 22E spaced from the handle 20E and a squeezed position with the end of the lever 22E spaced closer to the handle 20E than in the release position.

The manually powered surgical instrument 10E also includes a body portion 30E, extension 40E and a screw deployment portion 50E with a screw mount for receiving the self-tapping screw. The screw mount is rotatable to rotate the self-tapping screw into a pubic bone in a delivery rotation direction. The device optionally includes a screw shield 54E, irregular, tactile surface 52E and finger flanges 33E.

The finger flanges 33E may be grasped by the fingers of one hand and pulled up against the pubic bone, while the other hand is used to pump the lever 22D. Separation of the task of situating the bone screw and manually driving the instrument 10E is believed to afford greater control and afford precise delivery of the screw.

The bone screw mount is adapted to deliver the screw to the bone along a substantially straight bone screw delivery axis A′. The handle 20E preferably includes indicia 21E that is substantially in a region where a straight line drawn substantially along the bone screw delivery axis A′ would intersect the handle 20E.

The handle 22E and the bone screw deployment portion 50E of the surgical instrument 10E preferably project from a same side of the body portion 30E. Alternatively, the handle 22E and bone screw deployment portion 50E may project from opposite sides of the body portion 30E (e.g. similar to the shape shown in FIG. 23).

The surgical instrument 10E includes a manual drive that is operatively associated with the lever 22E to rotate the screw mount in the delivery rotation. Unlike prior art designs, the drive of the present invention is preferably capable of rotating the screw mount in the delivery rotation direction both when the lever 22E moves from the release position toward the squeezed position, and when the lever 22E moves from the squeezed position toward the release position.

A variety of different drives may be used with the manually driven instrument 10E shown in FIG. 24. One example of a suitable drive is shown schematically in FIG. 24. The drive includes pins 23E and 24E and slot 25E. A linear motion bar includes spiral surfaces 27E and the slot 25E. The linear motion bar is operatively associated with the handle 22E and biased by a spring 35E toward the release position. The surgical instrument 10E also includes a linear to rotational motion converter 29E that converts the linear motion of the linear motion bar to rotational motion of drive shaft 31E (e.g. in the direction of the arrow).

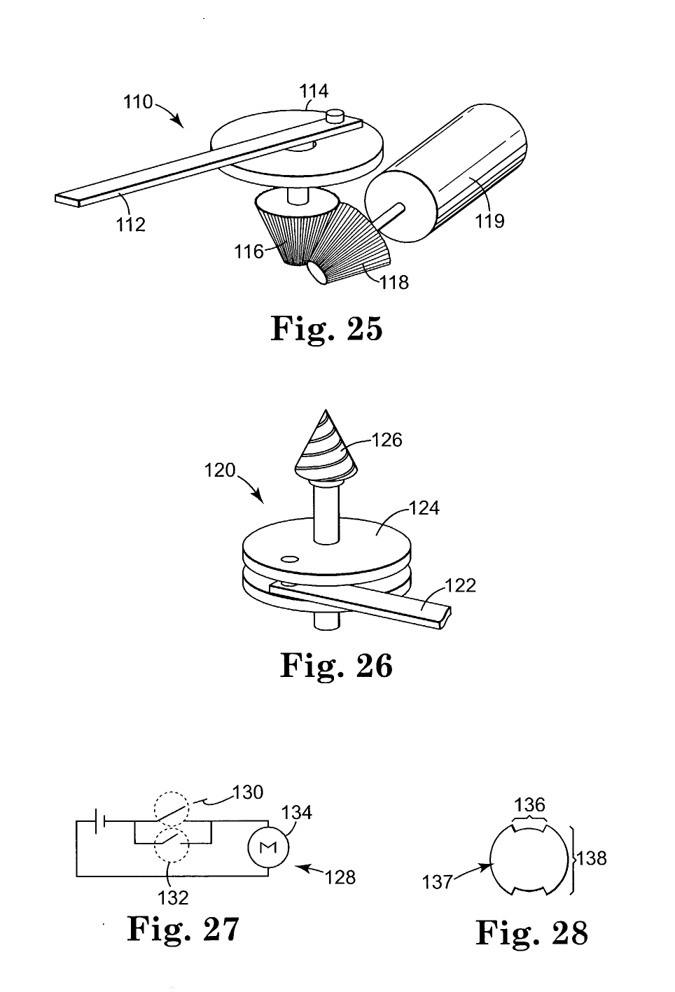

FIG. 25 schematically illustrates one example of a suitable linear to rotational converter comprising a right angle gear assembly 110. The converter includes a drive strip 112, an eccentric member 114, a drive gear 116 and a driven gear 118. The driven gear is attached to a rotating shaft 119.

FIG. 26 is a schematic illustration of another embodiment of motion converter 120. The converter comprises an eccentric 124, strip 122, and a rotational shaft for rotating the screw 126. A drawback of this concept is that it is possible that the eccentric 124 can stop in a position that is in line with the strip 122. In such an event, the shaft will not be able to rotate. FIGS. 27 and 28 schematically illustrate a microswitch assembly that may be used to address this issue in a motorized embodiment of this drive. The switch may be used to detect the rotational position of the eccentric and would only allow the eccentric disk to stop in an operational position.

The switch 128 includes a manual switch 130 and a safety switch 132. The switches 130 and 132 ensure that the motor 134 continues to run if the eccentric 124 attempts to stop in a position that is in line with the strip 122. Referring to FIG. 28, to accomplish this, element 137 includes regions 136 where the switch is closed, and regions 138 where the switch is open.

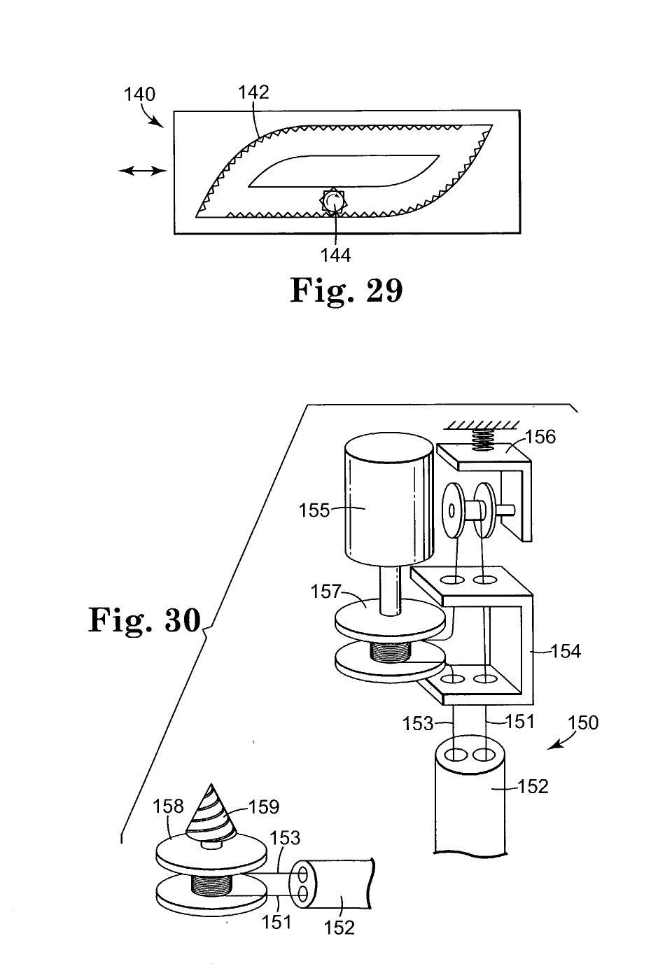

FIG. 29 illustrates another embodiment of linear to rotational motion converter. The converter includes a linear movable (see arrow) gear frame 140 with gear track 142 and rotational gear 144. When the frame 140 moves to the left the gear rolls along the gear track until it reaches the upper right hand comer of the frame 140. A spring (not shown) may bias the gear 144 into a secure fit against the gear track 142. As the gear 144 approaches the upper right hand comer of frame 140, leftward motion of the frame 140 stops and the frame 140 then moves to the right. The gear 144 detaches from the lower gear track 142 and engages the upper gear track 142. The gear will slide shortly along a non-geared surface and the spring before it engages the other track portion. The gear free surfaces can serve two roles i) the reciprocating motion is at its lowest force (the point of reversing the motion) and not driving the gear 144 and as such may prevent sticking or locking of the drive, and ii) shifting gears in motion requires good synchronization of the teeth. By providing the gear free surface, the gear 144 will smoothly transition to the gear track 142. Alternatively, structures may be used to push (cant) the frame 140 at each end of a stroke to accommodate the transition of the gear from the upper to the lower track.

FIG. 30 is a schematic illustration of another embodiment of drive 150 for use in an aspect of the present invention. The embodiment 150 utilizes a belt or continuous wire 151 and 153 system to drive a screw 159. The drive 150 utilizes a drive spool 157 that is driven by the motor 155 (or manually rotated shaft). The system includes a driven spool 158, a body with passages for the continuous line 151,153 and frames 154, 156. In use, the continuous line 151, 153 is driven off spool 157, threaded on driven spool 158 and then returns in a direction toward spool 157. This embodiment affords rotation of a remote element with a device configuration that minimizes the size of the body 152.

The take-up and unwind portions of the surgical instrument are substantially similar and comprise a spool over which a wire is tightly wound. At both ends of the spool the wire leaves the spool chamber that can be viewed as fixed. Since the spool is directly driven by the a drive element (e.g. optionally a motor), the wire will be wound up at bottom and unwound at the top, thereby providing continuous motion. A spring loaded tensioner secures friction between the spool and the wire. The wire is preferably wound at least 3 times, preferably 7-10 times to avoid slippage. A braided or profiled wire is preferred to enhance the friction between the wire and spool.

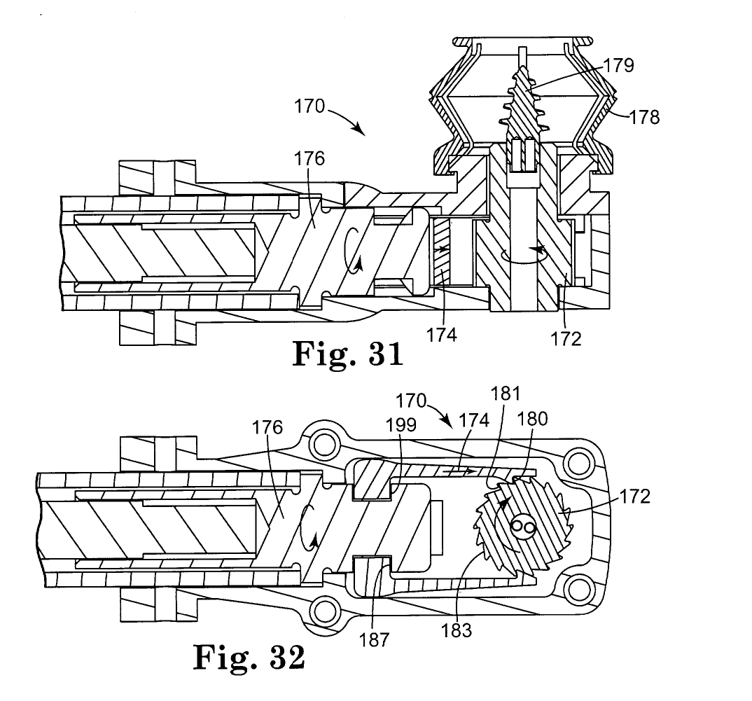

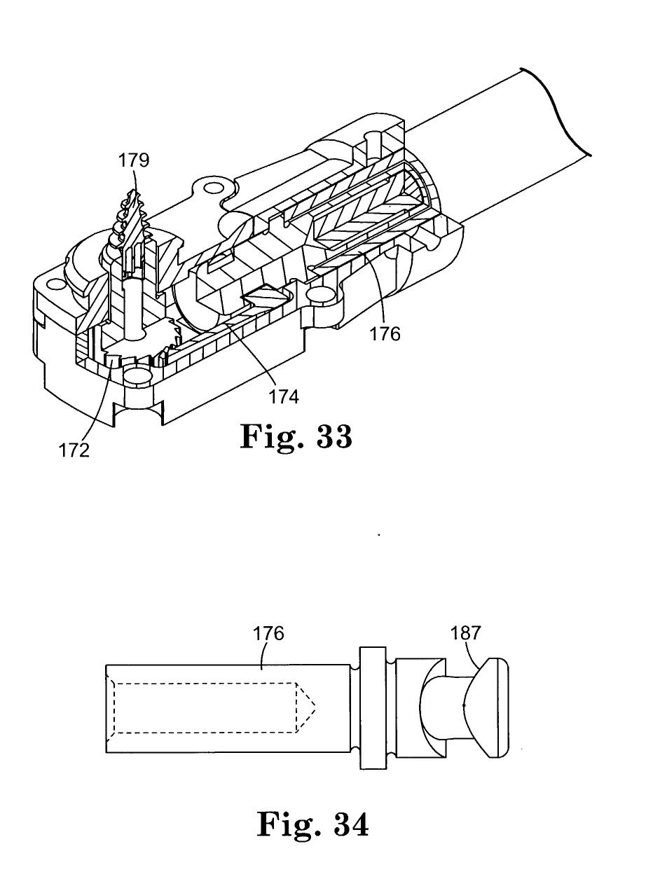

FIGS. 31 through 34 illustrate another embodiment of drive 170 according to the present invention. The drive 170 is suitable for use in a manually powered bone screw device for rotating a screw 179 into bone. The drive may optionally include a shield 178.

The drive 170 includes a rotatable shaft 176 with curved cam surfaces 187 (FIG. 34). The drive 170 includes a linearly movable member 174 having dual pawl arms. The linear movable member 174 includes cam follower surfaces 199 that are adapted to engage cam surfaces 187 to cause the linear movable member to reciprocate in a linear fashion. Alternatively, the rotatable shaft 176 and linear movable member 176 can simply be replaced with a linear reciprocating member having the dual pawl arms. Such an element may, for example, be driven by the manual drive components shown in FIG. 24 (excluding the spiral surfaces 27E and linear to rotational converter 29E).

Referring again to FIGS. 31-34, the drive shaft 176 is rotatable in the direction shown in FIG. 32. Rotation of the drive shaft 176 causes linear reciprocation of linearly movable member 174 in the direction shown in FIG. 32 (and in the opposite direction). In other words, the linearly movable member 174 is linearly movable in a proximal and a distal direction. The linear movable member 174 causes a ratchet wheel 172 to rotate in the direction of the arrow in FIG. 32 both when the linear movable member 174 moves proximally and when it moves distally. Rotating the ratchet wheel 172 both when the linear movable member 174 moves proximally and when it moves distally efficiently transfers power to the screw 179 and helps avoid the drawbacks of designs that require a user to manually pump or squeeze a lever in order to deliver a screw.

The pawl arms have specially shaped surfaces 180 that are adapted to engage specially shaped teeth surfaces 181 and 183 in ratchet wheel 172. The surface 180 of the pawl arm is adapted to engage a ledge surface 181 of a tooth of the ratchet wheel 172. This engagement occurs when the pawl arm drives the wheel 172 in the direction of the arrow in FIG. 32. On the return stroke (the non-power transmission stroke), the an angled surface of the pawl arm (generally opposite surface 180) is adapted to slide along release or angled surface 183 of the tooth of the wheel 172.

In another aspect, the present invention comprises a kit for treating a patient (e.g. for SUI). FIG. 19 shows a preferred kit comprising an inserter 10, at least one and preferably more bone anchors 84 (e.g. self-tapping screws) with associated sutures 83. The sutures 83 may be of a different kind, size or type (e.g. braided, monofilament, absorbable or non-absorbable). The sutures 83 may optionally include a threading tube to help thread the suture through the suture management tube of the instrument 10. The kit may also optionally include a screw loader 82 to assist in loading the base of the screw 84 into the anchor mount 69 without requiring the surgeon's gloves to touch the sharp, threaded surfaces of the self-tapping screw 84.

The kit is placed in packaging 12 and provided in a sterile condition to the surgeon. Additional elements may also be included in the kit for surgical convenience, for ease of manufacturing or sterilization, or for surgical requirements. For example, the kit may also include a sling material therein or attached thereto. As another example, the kit may include a plurality of different types of bone screws, such as bone screws with loop sutures, different length or types of sutures, or antimicrobial features such as those disclosed in U.S. Provisional Application Serial No. 60/295,330 filed Jun. 1, 2001.

Other accessories may also optionally be included in a kit according to the present invention. For example, a surgical drape specifically designed for urological procedures such as a sling procedure may be included in a kit of the present invention. Such a drape is disclosed in U.S. patent application Ser. No. 09/749,254, filed Dec. 27, 2001 (the entire contents incorporated herein by reference). Alternatively, one or more articles for objectively setting tension of the sling, such as those described in U.S. patent application Ser. No. 09/968,239, filed Oct. 1, 2001 (the entire contents of which are incorporated by reference) may be included in the kit.

### EXAMPLES OF METHODS

### Example 1

### Sling Procedure

Referring to FIGS. 7 through 9, surgical sling procedures for treating incontinence are generally illustrated. I.V. antibiotics may be administered prophylactically. The patient is placed in the lithotomy position and receives general, local or spinal anesthesia. After the patient has been prepped and draped, a Foley catheter (not shown) is placed in the bladder 6 and the balloon is inflated to approximately 20 cc. Pulling downward on the catheter, the balloon is palpated to identify the level of the bladder neck. Using this location as a reference point, the anterior vaginal wall of the vagina 2 is incised I to create exposure (e.g. from midurethra to bladder neck). A midline, inverted “U”, or “T” incision may alternatively be performed.

A defect of adequate size is optionally created to allow passage of the surgeon's index finger F alongside the inserter (e.g. 10 of FIGS. 1-6) in order to guide it into proper position on the posterior pubic bone 4. This is illustrated in FIG. 7. Tissue is laterally dissected to gain access to the retropubic space and to place the inserter 10 up against the bone 4. The extent of endopelvic fascia dissection is left to the surgeon's discretion. The inserter 10 is passed through this defect, preferably using the irregular surface 52 to guide the anchor delivery portion 50 into a therapeutically effective position (e.g. just below the bladder neck and approximately 2 cm lateral to the midline). Following the curvature of the pubic bone, the axis A of the inserter 10 is preferably positioned perpendicular to the posterior surface of the pubic bone 4. The surgeon may palpate the pubic bone (e.g. through the incision I or through the abdominal wall) and use reference indicia 26 to achieve proper orientation of the inserter 10 relative to the bone 4.

The surgeon firmly pulls the inserter upward against the bone surface to collapse the protective screw shield 54 and pierce the bone cortex with the tip of the screw 84. The surgeon then releases the lockout lever 24 of the inserter 10 while maintaining upward traction. The activation button 22 is depressed continuously for 5-15 seconds to drive the screw 84 completely into the bone 4. Notably, this action can be conducted with one handle, while the finger F (FIG. 7) of the other hand is used to help ensure no slippage of the anchor deployment portion 50 relative to the pubic bone 4.

A distinct change in motor tone indicates unimpeded rotation of the inserter's drill bit and full screw deployment. The activation button 22 is released and the inserter 10 is removed from the vagina 2. Caution is exercised to avoid knotting of the suture 83 due to actuation of the activation button 22 for an excessive amount of time.

The inserter 10 is then reloaded with a second screw from the kit. The positioning of the inserter 10 is repeated, but on the contralateral side of the urethral axis. The second screw is then placed.

The surgeon may then use a cystoscope to confirm integrity of the bladder 6 and urethra 8. A piece of sling material 1 is then prepared according to the surgeon's choice.

The surgeon then threads the sutures 83 from the first screw through one end of the sling material 1. The sutures 83 are tied and the knots are slid upward and posteriorly (behind the bone) to ensure juxtaposition of the sling end to the bone surface (FIG. 9). With the sling 1 lying beneath the urethra 8 just below the bladder neck, the surgeon may optionally place a small right-angle clamp between the sling 1 and the urethra 8. Using a second clamp, the sling material end is brought up to the pubic bone 4 to determine where along its length the sling 1 should be tied to the bone 4. With this measurement as a guide, the surgeon threads the remaining suture 83 pair (from the second screw) through the sling material 1 at the point determined. The surgeon then ties the sutures securely while maintaining the position of the clamp (not shown) between the sling 1 and the urethra 8. Any excess sling material is trimmed at this time. Optionally, the surgeon may associate the sling 1 with sutures 83 before inserting the bone screws with the inserter 10.

If desired, the distal aspect of the sling material 1 can optionally be secured to the periurethral fascia with absorbable sutures to prevent the graft from curling or migrating. The surgeon closes the vaginal wall incision I, preferably with a running absorbable suture.

It should be noted that the above surgical procedure may be altered in many ways according to surgeon custom or preference, or the needs of a particular patient. For example, some surgeons prefer no to dissect to the pubic bone. Instead, some surgeons place the screws at least partially through tissue (e.g. a portion of the vaginal wall or the endopelvic fascia) other than bone. Also, the above described procedure may be utilized in conjunction with concomitant pelvic floor repairs such as cystocele or prolapse repair.

### Ex. 2

### Manually Powered Device

In this embodiment, a manually powered device is used to implant the screws. For example, the instrument 10E (FIG. 24) or the instruments described in conjunction with FIGS. 31-34 may be used. In this embodiment, the fingers of one of the surgeon's hands are used to grasp finger flanges 33E and hold the device 10E in position relative to the pubic bone 4 while the surgeon's other hand is used to squeeze and release the lever 22E. Preferably, the drive of the device utilizes a drive mechanism that rotates the screw both when the lever 22E and when it is released. This helps minimize the coordination required of the surgeon to complete the procedure.

The method includes the steps of providing a self-tapping bone screw and an associated suture, and providing a manually powered surgical instrument comprising a handle, a manually movable lever having an end, the end of the lever being movable relative to the handle between a release position with the end of the lever spaced from the handle and a squeezed position with the end of the lever spaced closer to the handle than in the release position, and a screw mount that is rotatable to rotate the self-tapping screw into pubic bone in a delivery rotation direction. The method also includes the steps of placing the screw the in screw mount, creating a vaginal incision I (e.g. see FIG. 7), inserting a portion of the surgical instrument through the vaginal incision I, and manually manipulating the lever to rotate the screw mount in the delivery rotation direction both when the lever moves from the release position toward the squeezed position and when the lever moves from the squeezed position toward the release position.

### Ex. 3

### No Sling

The steps of examples 1 or 2 are repeated except that a sling 1 is not used. In this example, the sutures 83 associated with the screws 84 are threaded through vaginal tissue 2 or endopelvic fascia to reposition the tissue in a therapeutically effective position.

### Ex. 4

### Mandible Procedure

In this embodiment, the instrument 10 is used in the procedures for treating airway obstructions, sleep apnea and snoring as disclosed in U.S. Pat. No. 5,988,171.

### Ex. 5

### Sacrum Surgeries

The present invention may be utilized to place a bone anchor on a patient's sacrum. The sutures associated with the bone anchor may be used for a variety of different uses. For example, the sutures may be used to secure an electronic implant for treating urge incontinence.

Another use for a bone anchor attached to the sacrum is disclosed in U.S. patent application Publication No. U.S. 2002/0028980 (pub. date Mar. 7, 2002) (the entire contents incorporated herein by reference in its entirety). That publication describes a substantially straight bone anchor inserter for placing a bone screw in the sacrum during a sacral colpopexy procedure.

In the present invention, the inserter 10 is inserted through an incision in the patient's back (as opposed to an abdominal incision disclosed in the published application). The inserter 10 obtains access to the patient's sacrum without the requirement of an abdominal incision. The surgeon then has the option of performing the remaining steps of the procedure transvaginally, if desired.

### Ex. 6

### Soft Tissue and Spinal Procedures

The inserters described herein are suitable for orthopedic surgical procedures (e.g. soft tissue repair procedures). Orthopedic surgical procedures often include tying soft tissue such as ligament or cartilage to a bone screw with an associated suture. The inserters described herein are particularly suitable for such applications. especially at remote regions of the body. The inserters are particularly suitable for procedures that affords access to the bone on one side (e.g. an anterior side) but that requires placement of the bone anchor on the opposite side (e.g. the posterior side) of the bone.

Other applications include spinal surgeries where implants, cages or frames are often anchored directly to the patient's spine. Inserters according to the present invention afford approaching the patient's spine through a back incision and placing the bone anchor on the opposite side of the spine (e.g. the side facing the abdomen).

This invention may take on various modifications and alterations without departing from the spirit and scope thereof. Accordingly, it is to be understood that this invention is not to be limited to the above-described, but it is to be controlled by the limitations set forth in the claims and any equivalents thereof. It is also to be understood that this invention may be suitably practiced in the absence of any element not specifically disclosed herein.

Although the invention has been described in terms of particular embodiments and applications, one of ordinary skill in the art, in light of this teaching, can generate additional embodiments and modifications without departing from the spirit of or exceeding the scope of the invention. Accordingly, it is to be understood that the drawings and descriptions herein are proffered by way of example to facilitate comprehension of the invention and should not be construed to limit the scope thereof.

What is claimed is:

1. A surgical device for inserting a bone anchor comprising:
a handle,
a battery and motor assembly,
a bone anchor deployment portion having a first end portion and a second end portion with a bone anchor mount that is operatively associated with the battery and motor assembly, the bone anchor mount adapted to receive a bone anchor and to drive the bone anchor along a bone anchor delivery axis, the distance between an end of the bone anchor mount and the first end portion of the bone anchor deployment portion comprising the Bone Clearance Length,
a main body associated with the handle, and
the Bone Clearance Length is less than about 0.8 inches.

2. A surgical device according to claim 1 further including an extension portion projecting from the main body in a first direction, the extension portion including a body projection portion that projects from the main body, a bend and a portion that extends in a direction that is approximately ninety degrees relative to the direction of projection of the body projection portion.

3. A surgical device according to claim 2 wherein the bend comprises an arc having a radius of less than about 1.5 inches.

4. A surgical device according to claim 3 wherein the arc has a radius of less than about 1.2 inches.

5. A surgical device according to claim 1 wherein the bone anchor deployment portion is oriented to insert a bone anchor in a direction that is substantially toward the user of the device.

6. A surgical device according to claim 1 including a drive with a flexible shaft.

7. A surgical device according to claim 1 wherein the second end portion of the deployment portion faces the handle and the first end portion is opposite the second end portion, and the first end portion has an irregular surface in substantially the region where the bone anchor delivery axis would intersect the first end portion of the anchor deployment portion.

8. A surgical device according to claim 1 wherein the Bone Clearance Length is less than about 0.7 inches

9. A surgical device for inserting a bone anchor comprising:
a handle,
a battery and motor assembly,
a bone anchor deployment portion that is operatively associated with the battery and motor assembly, and that is adapted to receive a bone anchor and to drive the bone anchor along a bone anchor delivery axis,
a main body associated with the handle, and an extension portion projecting from the main body, the extension portion having an outer surface, the distance between the outer surface of the extension portion and the bone anchor delivery axis is the Insertion Depth Length, and
wherein the Insertion Depth Length is more than about 1.4 inches.

10. A surgical device according to claim 9 wherein the extension portion has a length that is more than about 1⅞ inches.

11. A surgical device according to claim 9 wherein the extension portion has a bend, and
the device includes a portion that extends in a direction that is approximately ninety degrees relative to the direction of projection of the extension portion.

12. A surgical device according to claim 9 wherein the bone anchor deployment portion is oriented to insert a bone anchor in a direction that is substantially toward the user of the device.

13. A surgical device according to claim 12 including a flexible drive shaft.

14. A surgical device according to claim 9 wherein the bone anchor deployment portion has a first end portion and a second end portion with a bone anchor mount that is operatively associated with the battery and motor assembly,
the second end portion of the deployment portion being oriented to face the handle, the first end portion is opposite the second end portion, and the first end portion has a palpable, irregular surface in substantially the region where the bone anchor delivery axis would intersect the first end portion of the anchor deployment portion.

15. A surgical device according to claim 11 wherein the bend comprises an arc having a radius of less than about 1.5 inches.

16. A manually powered surgical instrument for placing a self-tapping bone screw and an associated suture on the posterior portion of a pubic bone during a surgical procedure for treating incontinence, the instrument comprising:
a handle,
a manually movable lever having an end, the end of the lever being movable relative to the handle between a release position with the end of the lever spaced from the handle and a squeezed position with the end of the lever spaced closer to the handle than in the release position,
a body portion,
a screw mount for receiving the self-tapping screw, the screw mount being rotatable to rotate the self-tapping screw into pubic bone in a delivery rotation direction; and
a drive operatively associated with the lever to rotate the screw mount in the delivery rotation direction both when the lever moves from the release position toward the squeezed position and when the lever moves from the squeezed position toward the release position.

17. A surgical device according to claim 16 wherein the body portion includes a pair of finger flanges.

18. A surgical device according to claim 16 wherein the drive includes a double ratchet.

19. A surgical device according to claim 16 wherein the instrument is sized, shaped and arranged to be placed through a vaginal incision.

20. A surgical device according to claim 16 wherein the device includes a bone screw deployment portion that includes the screw mount, and
the handle and the bone screw deployment portion project from a same side of the body portion.

21. A surgical device according to claim 20 wherein bone screw mount is adapted to deliver the screw to the bone along a substantially straight bone screw delivery axis.

22. A manually powered surgical instrument for placing a self-tapping bone screw and an associated suture on the posterior portion of a pubic bone during a surgical procedure for treating incontinence, the instrument comprising:
a handle,
a manually movable lever having an end, the end of the lever being movable relative to the handle between a release position with the end of the lever spaced from the handle and a squeezed position with the end of the lever spaced closer to the handle than in the release position,
a body portion with a pair of finger flanges,
a bone screw deployment portion that includes a screw mount for receiving the self-tapping screw, the screw mount being rotatable to rotate the self-tapping screw into pubic bone in a delivery rotation direction;
the handle and the bone screw deployment portion project from a same side of the body portion, and
a drive operatively associated with the lever to rotate the screw mount in the delivery rotation direction.

23. A surgical device according to claim 22 wherein bone screw mount is adapted to deliver the screw to the bone along a substantially straight bone screw delivery axis.

24. A manually powered surgical instrument for placing a bone anchor and an associated suture on the posterior portion of a pubic bone during a surgical procedure for treating incontinence, the instrument comprising:
a handle,
a bone anchor deployment portion that includes a bone anchor mount for receiving the bone anchor;
a shield about the bone anchor mount,
a body portion between the handle and bone anchor deployment portion, the bone anchor deployment portion having a distal side and a proximal side opposite the distal side, the proximal side having the bone anchor mount and shield,
the bone anchor deployment portion being adapted to drive the bone anchor along a bone anchor delivery axis,
the distal side having a palpable, irregular surface in substantially the region where the bone anchor delivery axis would intersect the distal side of the anchor deployment portion.

25. A surgical device for inserting a bone anchor comprising:
a handle,
a drive including a flexible drive shaft,
means for rotating the flexible drive shaft,
a bone anchor deployment portion with a bone anchor mount that is operatively associated with the flexible drive shaft, the bone anchor mount adapted to receive a bone anchor and to insert the bone anchor along a bone anchor delivery axis,
a main body associated with the handle, and
an extension portion projecting from the main body in a first direction, the extension portion including a body projection portion that projects from the main body, a bend and a portion that extends in a direction that is approximately ninety degrees relative to the direction of projection of the body projection portion.

26. A surgical device according to claim 25 wherein the bend comprises an arc having a radius of less than about 1.5 inches.

27. A surgical device according to claim 25 wherein the drive includes a drive bevel gear associated with the flexible drive shaft.

28. A surgical device according to claim 27 wherein the bone anchor mount includes a driven bevel gear.

29. A surgical device according to claim 27 wherein the drive bevel gear includes compensation means for compensating for a reduction in length of the flexible drive shaft during rotation of the flexible drive shaft under a load.

30. A surgical device according to claim 29 wherein the compensation means comprises a channel in the drive bevel gear that affords axial movement of drive shaft relative to the drive bevel gear.

31. A surgical device according to claim 25 wherein the means for rotating the flexible drive shaft comprises a battery powered motor.

32. A surgical device according to claim 25 wherein the means for rotating the flexible drive shaft comprises manual powered elements capable of rotating the drive shaft without battery power.

33. A surgical device for inserting a bone screw comprising:
a handle,
a drive including a flexible, rotatable drive shaft that is bent about its axis about a radius,
means for rotating the flexible drive shaft,
a drive bevel gear capable of being rotated by the flexible drive shaft,
a bone anchor deployment portion with a bone anchor mount that includes a driven bevel gear capable of engaging the drive bevel gear to rotate the bone anchor mount in a screw delivery direction, the bone anchor mount adapted to receive a bone anchor and to insert the bone anchor along a bone anchor delivery axis, and
a main body associated with the handle.

34. A surgical device according to claim 33 wherein the drive bevel gear includes compensation means for compensating for a reduction in length of the flexible drive shaft during rotation of the flexible drive shaft under a load.

35. A surgical device according to claim 34 wherein the compensation means comprises a channel in the drive bevel gear that affords axial movement of the drive shaft relative to the drive bevel gear.

36. A surgical device according to claim 33 wherein the means for rotating the flexible drive shaft comprises a battery powered motor.

37. A surgical device according to claim 33 wherein the means for rotating the flexible drive shaft comprises manual powered elements capable of rotating the drive shaft without battery power.

38. A surgical device according to claim 33 wherein the radius is less than about 1.5 inches.

39. A surgical method for treating incontinence comprising the steps of:
providing a self-tapping bone screw and an associated suture, the screw being suitable for placement in the posterior portion of a pubic bone,
providing a manually powered surgical instrument comprising a handle, a manually movable lever having an end, the end of the lever being movable relative to the handle between a release position with the end of the lever spaced from the handle and a squeezed position with the end of the lever spaced closer to the handle than in the release position, and a screw mount that is rotatable to rotate the self-tapping screw into pubic bone in a delivery rotation direction,
placing the screw the in screw mount,
creating a vaginal incision,
inserting a portion of the surgical instrument through the vaginal incision, and
manually manipulating the lever to rotate the screw mount in the delivery rotation direction both when the lever moves from the release position toward the squeezed position and when the lever moves from the squeezed position toward the release position.

40. A surgical method according to claim 39 further including the step of tying a sling material to the suture associated with the bone screw.

41. A surgical method for treating incontinence comprising the steps of:
providing an anchor and an associated suture, the anchor being suitable for placement in a patient's pubic bone,
providing a surgical instrument comprising a handle, body and a bone anchor deployment portion that includes a bone anchor mount for receiving the bone anchor; the bone anchor deployment portion having a distal side and a proximal side opposite the distal side, the proximal side having the bone anchor mount and being arranged to face the handle, the distal side having a palpable, irregular surface,
placing the anchor the in anchor mount,
creating a vaginal incision,
inserting a portion of the surgical instrument through the vaginal incision,
using the irregular surface to orient the surgical instrument relative to the patient's pubic bone, and
inserting the bone anchor into the pubic bone.

42. A surgical procedure according to claim 41, further including the step of pushing against the irregular surface during the step of inserting the bone anchor into the pubic bone.
